(19) United States
(12) Patent Application Publication
(10) Pub. No.: US 2024/0399330 A1
(43) Pub. Date: Dec. 5, 2024

(54) METHOD FOR THE MANUFACTURE OF CORE-MEMBRANE CAPSULES, BY INTERFACIAL POLYMERIZATION

(71) Applicant: CAPSULAE, La Rochelle (FR)

(72) Inventors: Gisèle ONGMAYEB, Treillieres (FR); Juliette THIBAULT, Heric (FR); Laurent FONTAINE, Le Mans (FR); Véronique MONTEMBAULT, Le Mans (FR); Sagrario PASCUAL, Le Mans (FR)

(73) Assignee: CAPSULAE, La Rochelle (FR)

(21) Appl. No.: 18/691,553

(22) PCT Filed: Sep. 14, 2022
(86) PCT No.: PCT/EP2022/075584

§ 371 Date: Mar. 13, 2024

(30) Foreign Application Priority Data
Sep. 15, 2021 (FR) 2109664

(51) Int. Cl. B01J 13/16; C08G 69/28; C08G 69/42

(52) U.S. Cl. CPC B01J 13/16; C08G 69/28; C08G 69/42

(57) ABSTRACT

A method for the manufacture of core-membrane capsules, which manufacturing method includes: (i) a step of providing two immiscible solutions, namely: a first solution containing at least one first reactive molecule including at least two azlactone groups, and a second solution containing at least one second reactive molecule including at least two amine groups, and (ii) an interfacial polymerization step during which the immiscible solutions are mixed so that the reactive molecules react together by an interfacial polymerization reaction, on the surface of the droplets, to form the membrane including a polyamide polymer.

### TECHNICAL FIELD OF THE INVENTION

The present invention concerns the technical field of methods for the manufacture of core-membrane capsules.

It concerns in particular the methods for the manufacture of core-membrane capsules, by interfacial polymerization technique.

### STATE OF THE ART

The production of microcapsules from di- and polyisocyanates, and diethylene triamine (DETA), currently constitutes a method of choice for encapsulation manufacturers.

However, for reasons of toxicity, more drastic restrictions on the use of diisocyanates are being considered, which could extend to oligomers and polyisocyanates. Indeed, these molecules would be the cause of asthma in exposed workers.

There is therefore a need for new reactive molecules for the implementation of encapsulation techniques.

### PRESENTATION OF THE INVENTION

In order to remedy the aforementioned drawback of the state of the art, the present invention proposes to implement molecules with azlactone functionality as crosslinking agent in encapsulation methods by interfacial polymerization.

More particularly, according to the invention, a method is proposed for the manufacture of core-membrane capsules, which manufacturing method comprises the following successive steps:
(i) a step of supplying two immiscible solutions respectively containing reactive molecules capable of reacting together by an interfacial polymerization reaction to form a membrane comprising a polyamide polymer, namely:
   a first solution, preferably lipophilic, containing at least one first reactive molecule including at least two azlactone groups, and
   a second solution, preferably hydrophilic, containing at least one second reactive molecule including at least two amine groups,
one of said immiscible solutions, intended to form the core of said capsules, containing at least one active ingredient,
and
(ii) an interfacial polymerization step during which the immiscible solutions are mixed so that:
   the immiscible solution, containing said at least one active ingredient, forms a dispersed phase in the form of droplets intended to form the core of said capsules, and
   the other immiscible solution forms a continuous phase,
which reactive molecules react together by said interfacial polymerization reaction, on the surface of said droplets, to form said membrane comprising said polyamide polymer.

The present invention thus proposes new reactive molecules, for the implementation of encapsulation techniques, advantageously as an alternative to compounds from the isocyanate family.

Other non-limiting and advantageous characteristics of the product/method in accordance with the invention, taken individually or in all technically possible combinations, are as follows:
during the polymerization step, the first solution forms the dispersed phase and the second solution forms the continuous phase,
said at least two azlactone groups of the first reactive molecule each correspond to the following general formula (I)

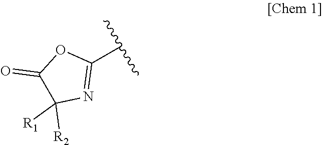

advantageously consisting of a derivative of a 5-(4H) oxazolone group,
in which R₁ and R₂ independently represent a (C₁-C₁₀)alkyl group, a (C₃-C₆) cycloalkyl group, an aryl group, an aryl (C₁-C₁₀)alkyl group, or a heterocyclic group;
   the first reactive molecule is selected from monomers including at least two azlactone groups, preferably from bis-azlactone, tris-azlactone or tetra-azlactone, or polymers of the polyazlactone type, advantageously from homopolymers, for example poly (2-vinyl-4,4-dimethylazlactone) (PVDM), or copolymers, for example among VDM-Styrene or VDM-DMA copolymers;
   the first reactive molecule is a bis-azlactone including two terminal azlactone groups which are connected by a hydrocarbon group, which can be interrupted by one or more oxygen or sulfur atoms, which hydrocarbon group advantageously consists of an aliphatic, linear or branched hydrocarbon group, comprising from 1 to 30, preferably from 1 to 20, carbon atoms which can be interrupted by one or more oxygen or sulfur atoms; said first reactive molecule advantageously corresponds to the following formula (II):

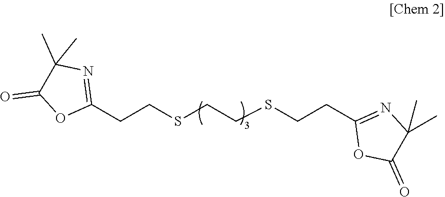

or the following formula (VII):

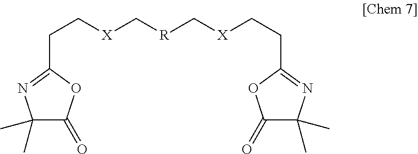

in which X represents S, O, CH₂
and R represents an aliphatic, linear or branched, hydrocarbon group, comprising from 1 to 20 carbon atoms which can be interrupted by one or more oxygen or sulfur atoms;
   the polyazlactone type polymers meet the following criteria: a molecular weight (also known as number average molar mass or Mn) ranging from 1,000 g/mol to 1,000,000 g/mol (or even from 1,000 g/mol to 100,000 g/mol) and/or a number of azlactone groups ranging from 20 to 70; preferably, the first solution contains a polyazlactone type polymer or a combination of at least two polyazlactone type polymers having different molecular weights; more preferably, said first reactive molecule corresponds to the following general formula (III)

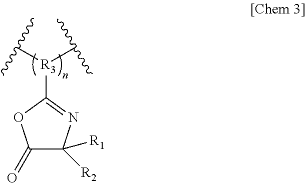

in which R₃ represents an aliphatic, linear or branched hydrocarbon group, comprising from 1 to 30, preferably from 1 to 20, carbon atoms which can be interrupted by one or more oxygen or sulfur atoms,

R₁ and R₂ independently represent a (C1-C10)alkyl group, a (C3-C6) cycloalkyl group, an aryl group, an aryl (C1-C10)alkyl group, or a heterocyclic group, and
n represents an integer from 20 to 70;
   the first reactive molecule advantageously corresponds to the following general formula (IV)

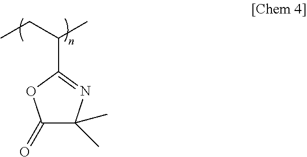

during the interfacial polymerization step, the mixture of the immiscible solutions is selected from a dripping operation of said first solution into said second solution or of said second solution into said first solution, or an emulsification operation;
the second reactive molecule is selected from molecules of a synthetic nature, for example hexamethylene diamine (HMDA), diethylene triamine (DETA), tris (2-aminoethyl) triamine (TREN) or polyethylene imine (PEI), or molecules of natural origin, for example chitosan;
the reactive molecules are selected from one of the following pairs: the first reactive molecule corresponding to formula (II)/DETA, the first reactive molecule corresponding to formula (II)/TREN, the first reactive molecule corresponding to formula (II)/HMDA, the first reactive molecule corresponds to formula (IV)/DETA, the first reactive molecule corresponds to formula (II)/chitosan, or the first reactive molecule corresponds to formula (II)/PEI;
the first solution is a lipophilic solution comprising a hydrophobic solvent, for example selected from benzyl benzoate, mineral oil, vegetable oil;
one of at least said immiscible solutions, preferably said two immiscible solutions, contains a surfactant;
the active ingredient is selected from lipophilic active ingredients and hydrophilic active ingredients,
said immiscible solutions are free of isocyanates, more preferably diisocyanates and/or polyisocyanates.

The present invention also concerns core-membrane capsules, resulting from a method according to the invention, including a polyamide membrane.

The core-membrane capsules advantageously have a diameter ranging from 50 nm to 5 mm, preferably from 5 to 100 μm.

Of course, the different characteristics, variants and embodiments of the invention can be associated with each other in various combinations as long as they are not incompatible or exclusive of each other.

### DETAILED DESCRIPTION OF THE INVENTION

The present invention thus concerns a method for manufacturing core-membrane capsules.

Such a manufacturing method is still commonly referred to as an encapsulation method or microencapsulation method.

By «encapsulation» or «microencapsulation», is meant in particular the method by which a product (advantageously liquid) is enclosed in a capsule.

By «capsules» is meant in particular particles (advantageously spherical) which are made up of a membrane (advantageously rigid) containing a core (advantageously liquid), and which thus constitute a reservoir system.

A capsule according to the invention constitutes a solid particle comprising a core (content), advantageously liquid, which is surrounded by a membrane (solid).

The term «membrane» means, according to the invention, a continuous wall (advantageously seamless) around the (advantageously liquid) encapsulated core.

Preferably, the membrane isolates and protects the core from the external environment and/or allows control of its release in a selected environment.

According to the invention, the membrane comprises a polyamide polymer.

A polyamide (PA) is a polymer containing amide functions.

Generally, the manufacturing method according to the invention uses reactive molecules carrying azlactone functions, in combination with reactive molecules carrying amine functions, to generate the polyamide polymer membrane by an interfacial polymerization reaction.

The azlactone function actually exhibits reactivity with the amine functions. The reaction of the amine function with the carbon carrying the ester function of the azlactone function cycle leading to the opening of this cycle.

This reaction leads to the creation of the amide bond, an example of which is illustrated in the following synthesis reaction (V) between an amine function and an azlactone type function:

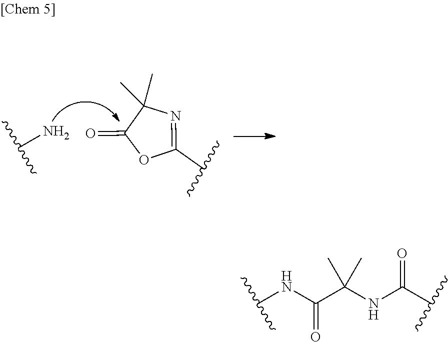

According to the invention, this reaction is taken advantage of in the context of the present invention, in encapsulation in order to form the polyamide membrane which envelops the core of the capsules.

### Manufacturing Method

According to the invention, the capsules are advantageously obtained by a manufacturing method comprising the following successive steps:
(i) a step of providing two immiscible solutions respectively containing reactive molecules capable of reacting together by an interfacial polymerization reaction to form a membrane comprising a polyamide polymer, and
(ii) an interfacial polymerization step during which the immiscible solutions are mixed so that the reactive molecules react together by said interfacial polymerization reaction, on the surface of droplets, to form said membrane comprising said polyamide polymer.

The two immiscible solutions, described in more detail below, are selected from:
a first solution, preferably lipophilic, containing at least one first reactive molecule including at least two azlactone groups, and
a second solution, preferably hydrophilic, containing at least one second reactive molecule including at least two amine groups.

Generally, an azlactone group is also referred to as an oxazolone group.

Preferably, said at least two azlactone groups of the first reactive molecule each correspond to general formula (I)

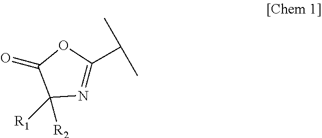

Such an azlactone group of general formula (I) according to the invention advantageously constitutes a derivative of a 5-(4H) oxazolone or 2-oxazolin-5-one group.

Such a group is further described in document FR-2 997 082 or document FR-3 054 235.

In this general formula (I), R₁ and R₂ independently represent a (C₁-C₁₀)alkyl group, a (C₃-C₆) cycloalkyl group, an aryl group, an aryl (C₁-C₁₀)alkyl group, or a heterocyclic group.

According to the present invention, the term «(C₁-C₁₀) alkyl» represents a saturated, linear or branched, hydrocarbon group, having from 1 to 10 carbon atoms, advantageously from 1 to 5 carbon atoms. By way of example, mention may be made of the methyl, ethyl, propyl, isopropyl, butyl, isobutyl, sec-butyl, tert-butyl, pentyl, neopentyl, isopentyl, tert-pentyl, hexyl, isohexyl, heptyl, octyl, nonyl and decyl groups.

A «(C₃-C₆) cycloalkyl group» represents a saturated, cyclic hydrocarbon group having 3 to 6 carbon atoms.

The term «aryl» represents a mono- or bicyclic aromatic hydrocarbon group comprising 6 to 10 carbon atoms. As an example, mention may be made of the phenyl and naphthyl groups.

By the term «aryl (C₁-C₁₀) alkyl», is meant an alkyl group having from 1 to 10 carbon atoms, advantageously from 1 to 5 carbon atoms as defined above and containing an aryl group as defined above.

The term «heterocyclic group» represents any saturated or unsaturated heterocycle, comprising from 3 to 7 carbon atoms and containing one to 3 heteroatoms selected from the group consisting of oxygen, nitrogen or sulfur. Mention may be made, for example, of piperidinyl, pyrrolidinyl, piperazinyl, pyridyl, piridinyl, imidazolyl, furyl, morpholinyl, oxetanyl, tetrahydrofuranyl, tetrahydropyranyl, tetrahydrothienyl, and thiazolyl groups.

Preferably, the groups R₁ and R₂ represent a methyl group.

More preferably, the first reactive molecule is selected from:
the monomers including at least two azlactone groups, preferably from bis-azlactone, tris-azlactone or tetra-azlactone, or
the polyazlactone polymers.

According to the «monomer» embodiment, the first reactive molecule is advantageously a bis-azlactone including two terminal azlactone groups which are connected by a hydrocarbon group, which can be interrupted by one or more oxygen or sulfur atoms.

Said hydrocarbon group advantageously consists of an aliphatic, linear or branched, hydrocarbon group, comprising from 1 to 30, preferably from 1 to 20, carbon atoms which can be interrupted by one or more oxygen or sulfur atoms.

In other words, the first reactive molecule of the bis-azlactone type advantageously corresponds to the following general formula (VI).

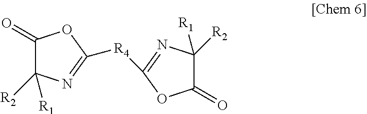

in which R₁ and R₂ are identical to general formula (I), and

R₄ represents an aliphatic, linear or branched, hydrocarbon group, comprising from 1 to 30, preferably from 1 to 20, carbon atoms which can be interrupted by one or more oxygen or sulfur atoms.

Such a first «monomeric» reactive molecule advantageously corresponds to the following formula (II):

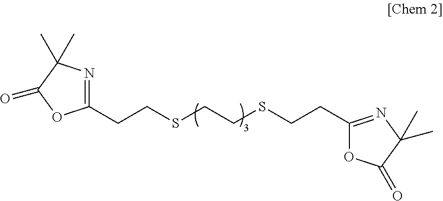

or the following formula (VII):

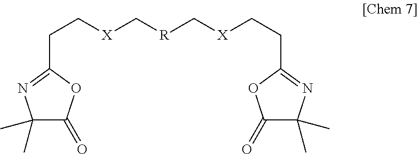

in which X represents S, O, CH₂

and R represents an aliphatic, linear or branched, hydrocarbon group, comprising from 1 to 20 carbon atoms which can be interrupted by one or more oxygen or sulfur atoms.

According to the «polymer» embodiment, the polyazlactone type polymers advantageously meet the following criteria:
a molecular weight (also called number average molar mass or Mn) ranging from 1,000 g/mol to 1,000,000 g/mol, or even from 1,000 g/mol to 100,000 g/mol, and/or
a number of azlactone groups ranging from 20 to 70.

The polymers advantageously have a dispersity from 1 to 6, preferably ranging from 2 to 6 for a polymer or ranging from 1 to 2 for a combination of polymers.

Preferably, the first solution contains:
a polymer of the polyazlactone type having a determined molecular weight, for example from 9,000 to 11,000 g/mol having a dispersity greater than 2, or
a combination of at least two polyazlactone polymers having different molecular weights (for example, respectively, from 15,000 to 17,000 g/mol and from 5,000 to 6,000 g/mol).

The polyazlactone type polymers are advantageously selected from:
the homopolymers, comprising monomer units including an azlactone group, for example poly (2-vinyl-4,4-dimethylazlactone) (PVDM), or
the copolymers, comprising at least one monomer unit including an azlactone group, for example among the VDM-styrene (2-vinyl-4,4-dimethylazlactone-styrene) or VDM-DMA (2-vinyl-4,4-dimethylazlactone-N,N-dimethylacrylamide) copolymers.

By «copolymer» is preferably meant a copolymer with a homogeneous structure, more preferably random or periodic.

According to the «homopolymer» embodiment, the first reactive molecule advantageously corresponds to the following general formula (III):

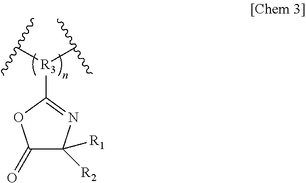

in which R₁ and R₂ are identical to general formula (I),

R₃ represents an aliphatic, linear or branched hydrocarbon group, comprising from 1 to 30, preferably from 1 to 20, carbon atoms which can be interrupted by one or more oxygen or sulfur atoms,

R₁ and R₂ independently represent a (C1-C10) alkyl group, a (C3-C6) cycloalkyl group, an aryl group, an aryl (C1-C10) alkyl group, or a heterocyclic group, and

n represents an integer from 20 to 70.

In this «polymer» embodiment, the first reactive molecule advantageously corresponds to the following general formula (IV):

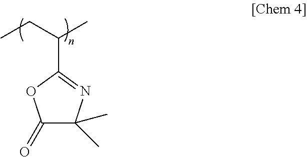

in which n advantageously represents an integer from 20 to 70.

Such a molecule is also called poly (2-vinyl-4,4-dimethylazlactone) (PVDM).

Still according to the invention, the first solution advantageously consists of a lipophilic solution comprising a hydrophobic solvent, for example selected from benzyl benzoate, mineral oil, vegetable oil.

Moreover, the second reactive molecule is advantageously selected from:
the molecules of a synthetic nature, for example hexamethylenediamine (HMDA), diethylenetriamine (DETA), tris(2-aminoethyl) triamine (TREN) or polyethyleneimine (PEI), or
the molecules of natural origin, for example chitosan.

By «chitosan», is advantageously expect the polysaccharide composed of the random distribution of D-glucosamine bonded in β-(1-4) (deacetylated unit) and N-acetyl-D-glucosamine (acetylated unit). It is produced by chemical (in an alkaline medium) or enzymatic deacetylation of chitin, the component of the exoskeleton of arthropods (crustaceans) or the endoskeleton of cephalopods (squid, etc.) or even of the wall of mushrooms. The chitosan advantageously has a degree of acetylation (DA) (percentage of acetylated units relative to the number of total units) less than 50%.

Generally and preferably, the reactive molecules are selected from one of the following pairs (first reactive molecule/second reactive molecule):
the first reactive molecule corresponding to formula (II)/DETA,
the first reactive molecule corresponding to formula (II)/TREN,
the first reactive molecule corresponding to formula (II)/HMDA,
the first reactive molecule corresponding to the formula (IV)/DETA,
the first reactive molecule corresponding to formula (II)/chitosan,
the first reactive molecule corresponding to formula (II)/PEI,
the (VDM-styrene) copolymer/DETA, or
the (VDM-DMA) copolymer/DETA.

Still generally, at least one of said immiscible solutions, preferably said two immiscible solutions, contains a surfactant.

The presence of at least one surfactant is useful to facilitate the penetration of drops from the dispersed phase into the continuous phase.

Such a surfactant is for example selected from:
the non-ionic surfactants, for example Marlipal 24/70, Tween 20, Tween 80, or
the ionic surfactants, for example SDS.

Still generally, one of said immiscible solutions, intended to form the core of said capsules, contains at least one active ingredient.

The active ingredient is advantageously selected from lipophilic active ingredients and hydrophilic active ingredients.

More preferably, said at least one active ingredient is selected from:
the hydrophilic active ingredients, for example B vitamins, gels (Aloe vera), enzymes, hemp extract, and/or
the lipophilic active ingredients, for example vitamins A, C, D or E, omega-rich oils, essential oils, perfumes, mineral oils, vegetable oils, phase change materials, plant extracts, essential oils, marine oils, krill oils, dyes, flavorings, perfumes, flavoring compositions for applications for tobacco and e-liquids, solid microparticles made of wax and containing a lipophilic or hydrophilic active ingredient.

The immiscible solutions are then mixed to carry out the interfacial polymerization step.

By «interfacial polymerization» is meant in particular a polymerization occurring at the interface between two immiscible solutions, resulting in a polymer which is constrained at the interface.

In this context, during said interfacial polymerization step, the immiscible solutions are mixed so that:
an immiscible solution, containing said at least one active ingredient, forms a dispersed phase in the form of droplets intended to form the core of said capsules, and
the other immiscible solution forms a continuous phase.

The reactive molecules then react together by said interfacial polymerization reaction, on the surface of said droplets, to form said membrane comprising said polyamide polymer.

To obtain the dispersed/continuous phases, the mixture of immiscible solutions is advantageously selected from a dripping operation:
of said first solution in said second solution, or
of said second solution in said first solution.

The mixture of immiscible solutions can also consist of an emulsification operation:
of said first solution in said second solution, or
of said second solution in said first solution.

According to a preferred embodiment, during the polymerization step:
the first solution (containing said at least one first reactive molecule including at least two azlactone groups) forms the dispersed phase, and
the second solution (containing at least one second reactive molecule including at least two amine groups) forms the continuous phase.

Said interfacial polymerization step is still advantageously implemented with at least one of the following conditions:
a pH of the continuous phase ranging from 6 to 12,
a temperature ranging from 20° C. to 50° C.,
a mechanical agitation (for example 100 to 150 revolutions per minute).

The generated core-membrane capsules are then recovered from the continuous phase and transferred to aqueous solution, or dried.

Generally and according to a preferred embodiment, said immiscible solutions are free of isocyanates, more preferably diisocyanates and/or polyisocyanates.

In other words, the reactive molecules are free of isocyanates, more preferably diisocyanates and/or polyisocyanates.

### Core-Membrane Capsules

The method according to the invention makes it possible to obtain core-membrane capsules including a polyamide membrane.

Such core-membrane capsules advantageously have a diameter ranging from 50 nm to 5 mm, preferably from 5 to 100 μm.

Of course, various other modifications can be made to the invention within the scope of the appended claims.

The present invention is further illustrated in the context of the Examples below.

### EXAMPLES

### Example 1

### Preparing Solutions

Solution A: a solution of 0.5 g of Marlipal 24/70 supplemented to 100 g with water was prepared. 24 g of this solution were taken and placed in a beaker. 6 g of HMDA was added. The solution was stirred for 10 min.

Solution B: 0.2 g of bis-azlactone and less than 0.05 g of hydrophobic red dye were solubilized in 0.8 g of benzyl benzoate. The solution was placed in an Eppendorf tube then placed in a water bath with magnetic stirring at a temperature of 50° C.

The bis-azlactone corresponds to the following formula (II):

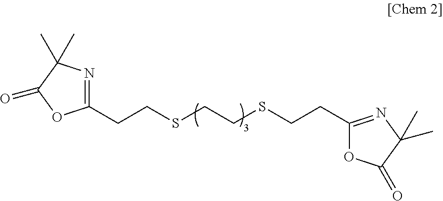

Drip Solution B into Solution A

Solution B was drawn into a syringe. The syringe was then placed 3 cm above solution A. Through a cone, the drops of solution B fell into solution A. Solution A was stirred throughout for 1 hour. The capsules were then removed, washed with ethanol and redissolved in water or dried at room temperature.

### Analysis of the Obtained Microcapsules

The analysis is then carried out by observation using a binocular magnifying glass. The capsule membrane is visible and the average capsule diameter is 2.5 mm. The capsules could be washed and dried. The dry membrane is transparent and leaves the liquid core.

The conditions of this example 1 are further developed in table 1 below

TABLE 1

| Setting | Conditions |
|---|---|
| Aqueous solution | Total volume: 10-100 mL |
| Crosslinking agent with amine | Concentration: 10-30% by weight |
| functions | |
| Water | |
| Surfactant: Marlipal 24/70 or | Concentration: 0.5% by weight |
| SDS | |
| Oily solution | Total volume: 1-5 mL |
| Bis-azlactone | Concentration: 10-30% by weight |
| Benzyl benzoate | |

| Dripping setting | Conditions |
|---|---|
| Cone diameter | 200 μm |
| Stirring | Orbital or mechanical |
| Temperature | 20-50° C. |

### Example 2

### Preparing Solutions

Solution A: a solution of 1 g of hydroxypropyl methylcellulose supplemented to 100 g with water was prepared. 27 g of this solution were taken and placed in a 30 mL centrifugation tube. 3 g of DETA was added. The solution was stirred for 10 min.

Solution B: 0.2 g of PVDM and less than 0.05 g of hydrophobic red dye were solubilized in 0.8 g of benzyl benzoate. The solution was placed in an Eppendorf tube then placed in a water bath with magnetic stirring at a temperature of 50° C.

The PVDM has the following characteristics:
Mn: 16500 g/mol,
Dispersity: 1.9
   Dripping Solution B into Solution A

Solution B was drawn into a syringe. A cone was placed at the end of the syringe then plunged 0.5 cm below the surface of solution A. Through a cone, the drops of solution B slowly fall into solution A. When the dropping was completed, the tube was stirred on a rotary shaker at 20 revolution per minute for 1 h. The capsules were then removed, washed with ethanol and redissolved in water or dried at room temperature.

### Analysis of the Obtained Microcapsules

The analysis is then carried out by observation using a binocular magnifying glass. The capsule membrane is visible and the average diameter of the capsule is 3 mm. The capsules could be washed and dried, revealing liquid core capsules and transparent membrane.

The conditions of Example 2 are further developed in Table 2 below.

TABLE 2

| Settings | Conditions |
|---|---|
| Aqueous solution | Total volume: 30 mL |
| Crosslinking agent with amine | Concentration: 10-30% by weight |
| functions | |
| Water | Concentration: 1% by weight |
| Thickener: | |
| hydroxypropylmethylcellulose | |
| Oily solution | Total volume: 1-2 mL |
| Bis-azlactone | Concentration: 10-30% by weight |
| Benzyl benzoate | |

| Dripping Settings | Conditions |
|---|---|
| Cone diameter | 200 μm |
| Stirring | Rotary |
| Temperature | Ambient |

### OTHER EXAMPLES

Core-membrane microcapsules were further synthesized in the combinations and conditions recalled in Table 3 below.

TABLE 3

| First reactive | Second reactive | |
|---|---|---|
| molecule | molecule | Conditions |
| Bis-azlactone | DETA | Dripping: |
| | | 20% by weight of bis-azlactone in |
| | | benzyl benzoate with 0.5% by |
| | | weight of Span 20 |
| | | 10% by weight of DETA in water |
| | | with 0.5% by weight of Marlipal |
| | | 24/70. |
| Bis-azlactone | DETA | Dripping: |
| | | 20% by weight of bis-azlactone in |
| | | benzyl benzoate with 0.5% by |
| | | weight of Span 20 |
| | | 10% by weight of TREN in water |
| | | with 0.5% by weight of Marlipal |
| | | 24/70. |
| Bis-azlactone | TREN | Dripping: |
| | | 20% by weight of bis-azlactone in |
| | | benzyl benzoate with 0.5% by |
| | | weight of Span 20 |
| | | 10% by weight of TREN in water |
| | | with 0.5% by weight of Marlipal |
| | | 24/70. |
| Bis-azlactone | HMDA | Dripping: |
| | | 20% by weight of bis-azlactone in |
| | | benzyl benzoate with 0.5% by |
| | | weight of Span 20 |
| | | 10% by weight of HMDA in water |
| | | with 0.5% by weight of Marlipal |
| | | 24/70. |
| PVDM | DETA | Dripping: |
| (Mn: 3500 g/mol | | 20% by weight of PVDM in benzyl |
| Dispersion: 1.34) | | benzoate |
| | | 10% by weight of DETA in water |
| | | with Methocel K4M at 1% by |
| | | weight |
| PVDM | DETA | Dripping: |
| (Mn: 5600 g/mol | | 20% by weight of PVDM in |
| Dispersion: 1.47) | | benzyl benzoate |
| | | 10% by weight of DETA in |
| | | water with Methocel K4M at 1% |
| | | by weight |
| PVDM | DETA | Dripping: |
| (Mn: 9000 g/mol | | 20% by weight of PVDM in |
| Dispersion: 1.76) | | benzyl benzoate |
| | | 10% by weight of DETA in |
| | | water with Methocel K4M at 1% |
| | | by weight |
| PVDM | DETA | Dripping: |
| (Mn: 7000 g/mol | | 20% by weight of PVDM in |
| Dispersion: 5.18) | | benzyl benzoate |
| | | 10% by weight of DETA in |
| | | water with Methocel K4M at 1% |
| | | by weight |
| PVDM | DETA | Dripping: |
| (Mn: 16500 g/mol | | 20% by weight of PVDM in |
| Dispersion: 5.18) + | | benzyl benzoate (50% PVDM |
| PVDM | | 16500 g/mol and 50% PVDM |
| (Mn: 5600 g/mol | | 5600 g/mol) |
| Dispersion: 1.47) | | 10% by weight of DETA in |
| | | water with Methocel K4M at 1% |
| | | by weight |
| (VDM-styrene) | DETA | Dripping: |
| copolymer | | 10% by weight of (VDM- |
| | | styrene) copolymer in benzyl |
| | | benzoate |
| | | 10% by weight of DETA in |
| | | water with SDS 0.5% by weight |
| (VDM-DMA) | DETA | Dripping: |
| copolymer | | 10% by weight of (VDM-DMA) |
| | | copolymer in benzyl benzoate |
| | | 10% by weight of DETA in |
| | | water with SDS at 0.5% by |
| | | weight |

1. A method for the manufacture of core-membrane capsules, which manufacturing method comprises the following successive steps:
(i) a step of supplying two immiscible solutions respectively containing reactive molecules capable of reacting together by an interfacial polymerization reaction to form a membrane comprising a polyamide polymer, namely:
a first solution containing at least one first reactive molecule including at least two azlactone groups, and
a second solution containing at least one second reactive molecule including at least two amine groups,
one of the immiscible solutions, intended to form the core of the capsules, containing at least one active ingredient,
and
(ii) an interfacial polymerization step during which the immiscible solutions are mixed so that:
the immiscible solution, containing the at least one active ingredient, forms a dispersed phase in the form of droplets intended to form the core of the capsules, and
the other immiscible solution forms a continuous phase,
which reactive molecules react together by the interfacial polymerization reaction, on the surface of the droplets, to form the membrane comprising the polyamide polymer.

2. The method for the manufacture of core-membrane capsules, according to claim 1, wherein, during the polymerization step:
the first solution forms the dispersed phase, and
the second solution forms the continuous phase.

3. The method for the manufacture of core-membrane capsules, according to claim 1, wherein the at least two azlactone groups of the first reactive molecule each correspond to the following general formula (I). 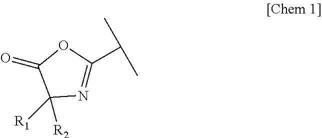
advantageously consisting of a derivative of a 5-(4H) oxazolone group,
in which R₁ and R₂ independently represent a (C₁-C₁₀)alkyl group, a (C₃-C₆) cycloalkyl group, an aryl group, an aryl(C₁-C₁₀)alkyl group, or a heterocyclic group.

4. The method for the manufacture of core-membrane capsules, according to claim 1, wherein the first reactive molecule is selected from:
the monomers comprising at least two azlactone groups, or
the polymers of the polyazlactone type.

5. The method for the manufacture of core-membrane capsules, according to claim 4, wherein the first reactive molecule is a bis-azlactone including two terminal azlactone groups which are connected by a hydrocarbon group, which can be interrupted by one or several oxygen or sulfur atoms,
which hydrocarbon group consists of an aliphatic, linear or branched, hydrocarbon group comprising from 1 to 30 carbon atoms which can be interrupted by one or more oxygen or sulfur atoms.

6. The method for the manufacture of core-membrane capsules, according to claim 5, wherein the first reactive molecule corresponds to the following formula (II): 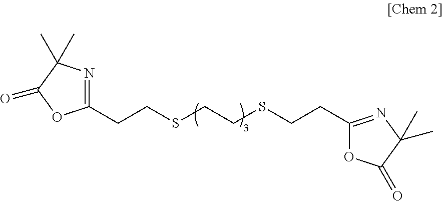
or the following formula (VII): 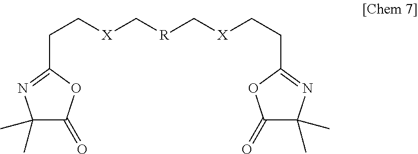
in which X represents S, O, CH₂
and R represents an aliphatic, linear or branched, hydrocarbon group, comprising from 1 to 20 carbon atoms which can be interrupted by one or more oxygen or sulfur atoms.

7. The method for the manufacture of core-membrane capsules, according to claim 4, wherein the polyazlactone polymers meet the following criteria:
a molecular weight ranging from 1,000 g/mol to 1,000,000 g/mol, and/or
a number of azlactone groups ranging from 20 to 70.

8. The method for the manufacture of core-membrane capsules, according to claim 7, wherein the first reactive molecule corresponds to the following general formula (III): 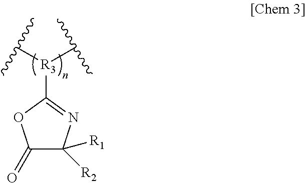
in which R₃ represents an aliphatic, linear or branched, hydrocarbon group comprising from 1 to 30 carbon atoms which can be interrupted by one or more oxygen or sulfur atoms, R1 and R2 independently represent a (C1-C10)alkyl group, a (C3-C6) cycloalkyl group, an aryl group, an aryl(C1-C10)alkyl group, or a heterocyclic group, and n represents an integer from 20 to 70.

9. The method for the manufacture of core-membrane capsules, according to claim 8, wherein the first reactive molecule corresponds to the following general formula (IV):

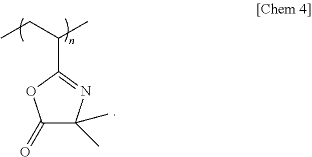

10. The method for the manufacture of core-membrane capsules, according to claim 1, wherein, during the interfacial polymerization step, the mixture of immiscible solutions is selected from a dripping operation or an emulsification operation.

11. The method for the manufacture of core-membrane capsules, according to claim 1, wherein, the second reactive molecule is selected from:
the molecules of a synthetic nature, or
the molecules of natural origin.

12. The method for the manufacture of core-membrane capsules, according to claim 11, wherein the reactive molecules are selected from one of the following pairs:
the first reactive molecule corresponding to formula (II)/DETA,
the first reactive molecule corresponds to formula (II)/TREN,
the first reactive molecule corresponds to formula (II)/HMDA,
the first reactive molecule corresponds to the formula (IV)/DETA,
the first reactive molecule corresponds to formula (II)/chitosan, or
the first reactive molecule corresponds to formula (II)/PEI.

13. The method for the manufacture of core-membrane capsules, according to claim 1, wherein at least one of the immiscible solutions contains a surfactant.

14. The method for the manufacture of core-membrane capsules, according to claim 1, wherein the immiscible solutions are free of isocyanates.

15. A core-membrane capsules, resulting from a method according to claim 1, including a polyamide membrane.
